(19) United States
(12) Patent Application Publication
(10) Pub. No.: US 2019/0282642 A1
(43) Pub. Date: Sep. 19, 2019

(54) Method of Producing Cannabidiol Derived Products

(71) Applicant: Christine Meeusen, Merced, CA (US)

(72) Inventor: Christine Meeusen, Merced, CA (US)

(21) Appl. No.: 16/428,179

(22) Filed: May 31, 2019

Related U.S. Application Data
(62) Division of application No. 15/842,662, filed on Dec. 14, 2017.
(60) Provisional application No. 62/435,007, filed on Dec. 15, 2016.

(51) Int. Cl. A61K 36/185; A01C 21/00; A61K 31/05; A01D 45/00; A61K 47/46; A01G 22/00; A01C 14/00

(52) U.S. Cl. CPC A61K 36/185; A01C 21/00; A61K 31/05; A01D 45/00; A61K 2236/37; A01G 22/00; A01C 14/00; A01C 21/005; A61K 47/46

(57) ABSTRACT

Methods are provided for producing cannabidiol (CBD)-based products in accordance with cycles of the moon and a plurality of biodynamic rituals. In some embodiments, the phase of the moon may be correlated with positions of the moon among the twelve zodiac signs, and the positions used to guide in the cultivation and harvest of cannabis plants. In some embodiments, the phase of the moon and the corresponding astrological phase that the moon is moving through may be used to determine when to plant seeds, when/how to water and fertilize, when to take cuttings, and the right time to harvest mature cannabis plants to produce optimal CBD-derived products. It is contemplated that farming cannabis with consideration of moon cycles may result in healthier plants, increased yields and superior products.

### PRIORITY

This application is a continuation of U.S. patent application Ser. No. 15/842,662, filed Dec. 14, 2017, which claims the benefit of and priority to U.S. Provisional Application No. 62/435,007, filed Dec. 15, 2016, both entitled “Method Of Producing Cannabidiol Derived Products,” and are incorporated by reference herein in their entirety.

### FIELD

The field of the present disclosure generally relates to cannabis-based products. More particularly, the field of the invention relates to a method for producing CBD derived products according to cycles of the moon and a plurality of biodynamic ritual.

### BACKGROUND

In the past 25 years, scientific research into cannabis has demonstrated the existence of naturally occurring compounds and receptors in the brain and body broadly referred to as the endogenous cannabinoid system. More specifically, cannabinoids—neurotransmitters natively produced in the body—have been found to impact regulation of one's mood, pain, memory, appetite, and immune response.

Two of the main active compounds in cannabis are tetrahydrocannabinol (THC) and cannabidiol (CBD), among many others. THC is comparable to the naturally occurring neurotransmitter anandimide (which may also be found in chocolate) and is generally considered to be responsible for marijuana's psychoactive effects. Notably, CBD is non-psychoactive and may have a range of beneficial effects, including as an anti-inflammatory and anti-anxiety agent.

Scientific study has also demonstrated that the endocannabinoid system may have broad impact throughout the body and mind, especially considering that cannabis contains a large range of specific compounds that impact this system, in varying ratios. Accordingly, marijuana's effects on the body may be very subjective, and differ according to the strain and dosage ingested.

Despite harsh laws against its use, recent studies indicate that marijuana is the third most popular recreational drug in America (behind only alcohol and tobacco), and has been used by nearly 100 million Americans. Furthermore, recent polls indicate that a majority of Americans now support legalization of marijuana and support is growing each year. Several states have already decriminalized marijuana, legalized it for medical use, or legalized it completely. As the cultural perception of cannabis becomes less negative, production is bound to increase.

What is needed, therefore, is a new method of producing high quality CBD derived products using improved harvesting techniques that take into account moon cycles and a plurality of secular, biodynamic rituals.

### SUMMARY

Methods are provided for producing CBD-based products in accordance with cycles of the moon and a plurality of biodynamic rituals. In some embodiments, the phase of the moon may be correlated with positions of the moon among the twelve zodiac signs, and the positions used to guide in the cultivation and harvest of cannabis plants. More specifically, the phase of the moon and the corresponding astrological phase that the moon is moving through may be used to determine when to plant seeds, when/how to water and fertilize, when to take cuttings, and the right time to harvest mature cannabis plants to produce optimal CBD-derived products. It is contemplated that farming cannabis with consideration of moon cycles may result in healthier plants, increased yields and superior products. The methods described herein may be performed indoors or outdoors without exceeding beyond the spirit and scope of the present disclosure.

In an exemplary embodiment, a method for producing a CBD-derived product comprises: determining a moon phase; performing at least one ritual on the first day of a moon cycle; communicating one or more incantations; cleansing each participant of the CBD production; distributing cannabis into one or more containers; extracting CBD from the cannabis; and forming the CBD-derived product.

In another exemplary embodiment, determining the moon phase includes identifying an occurrence of a full moon. In another exemplary embodiment, determining the moon phase includes establishing the moon phase as any one of waning crescent, third quarter, waning Gibbous, full moon, waxing Gibbous, first quarter, and waxing crescent. In another exemplary embodiment, determining the moon phase further comprises identifying positions of the moon among the twelve zodiac signs, and using the positions to guide in the cultivation and harvest of the cannabis plants.

In another exemplary embodiment, performing the at least one ritual includes considering a first zodiac sign prior to the at least one ritual. In another exemplary embodiment, performing the at least one ritual includes performing a biodynamic ritual that comprises lighting bundles of sage. In another exemplary embodiment, performing the at least one ritual includes performing the biodynamic ritual to bless a work environment. In another exemplary embodiment, cleansing comprises lighting a bundle of sage and sweeping the sage over any of a plurality of bottles and ingredients that may include a high-proof alcohol.

In another exemplary embodiment, distributing comprises disposing appropriate dosages of cannabis into one or more preparation jars that are suitable for production of one or more CBD derived products. In another exemplary embodiment, extracting is comprised of a solvent-based extraction method that includes submerging the cannabis in a plurality of suitable solvents to produce a resultant CBD concentrate, and straining the resultant CBD concentrate of plant matter and solvents. In another exemplary embodiment, extracting is comprised of a CO2 extraction method that includes subjecting the cannabis to carbon dioxide under high pressure and very low temperature so as to produce a resultant CBD concentrate, and straining the resultant CBD concentrate of plant matter. In another exemplary embodiment, extracting includes infusing the cannabis with olive oil so as to produce a resultant CBD concentrate.

In an exemplary embodiment, a method for producing CBD-derived products comprises: determining an instance of a new moon; planting cannabis cuttings during a beginning of a first quarter of the lunar cycle; performing at least one biodynamic ritual during a third quarter of the lunar cycle; maintaining cannabis plants during a fourth quarter of the lunar cycle; celebrating rest and reflection through one or more biodynamic rituals during the third quarter and the fourth quarter; and harvesting mature cannabis plants under the glow of the full moon during the third quarter.

In another exemplary embodiment, determining includes correlating the lunar cycle with the twelve zodiac signs so as to guide in the cultivation and harvest of the mature cannabis plants. In another exemplary embodiment, maintaining cannabis plants includes implementing any of watering, fertilizing, and managing pests.

In another exemplary embodiment, the method further comprises extracting a CBD concentrate from harvested mature cannabis plants. In another exemplary embodiment, extracting includes submerging cannabis in one or more suitable solvents and straining of plant matter and solvents to produce the CBD concentrate. In another exemplary embodiment, extracting includes subjecting the cannabis to carbon dioxide under high pressure and very low temperature and straining of plant matter to produce the CBD concentrate. In another exemplary embodiment, extracting includes infusing the cannabis with olive oil and straining of plant matter to produce the CBD concentrate. In another exemplary embodiment, the method further comprises incorporating a suitable quantity of the CBD concentration into any of a plurality of desired CBD-based products.

### BRIEF DESCRIPTION OF THE DRAWINGS

The drawings refer to embodiments of the present disclosure in which:

FIG. 1 illustrates an exemplary method of producing CBD derived products, taking into account moon cycles and a plurality of secular, biodynamic rituals;

FIG. 2 illustrates moon phases for harvesting CBD derived products using according to one embodiment of the present disclosure; and

FIG. 3 illustrates zodiac signs that correspond to a plurality of moon phase ranges according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first element,” may be made. However, the specific numeric reference should not be interpreted as a literal sequential order but rather interpreted that the “first element” is different than a “second element.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the item's “about,” “approximately,” or “substantially” for any numerical values or ranges indicate a suitable dimensional tolerance that allows the part or collection of components to function for its intended purpose as described herein.

In general, the present disclosure describes methods for producing CBD derived products according to cycles of the moon and a plurality of biodynamic rituals. More specifically, embodiments of the present disclosure utilize the phase of the moon and the corresponding astrological phase that the moon is moving through, so as determine when to plant seeds, take cuttings, when/how to water and fertilize, and the right time to harvest plants to produce CBD derived products. It is envisioned that farming marijuana with consideration of moon cycles may result in healthier plants, tastier buds and increased yields. It should be understood that methods described herein may be performed indoors or outdoors without exceeding beyond the spirit and scope of the present disclosure.

As an introductory matter, reference to biodynamic rituals (or ceremonies) herein indicates one or more activities that may be performed so as to promote understanding and appreciation of soil fertility and plant growth, such that spiritual, astrological and mystical perspectives are emphasized. As such, it should be understood that reference to biodynamic ritual should not be limited to any specific ritual or style thereto. By way of non-limiting example, a planting calendar may be implemented. In such an approach, lunar and astrological influences on soil and plant development may be considered. More specifically, as discussed herein, one may choose to plant, cultivate or harvest various crops based on both the phase of the moon and the zodiacal constellation the moon is passing through. On may also consider, for example, whether the crop is the root, leaf, flower, or fruit of the plant.

In one embodiment, a method for producing CBD derived products is disclosed, comprising: determining a moon phase; performing a first biodynamic ritual on the first day of a moon cycle; communicating a first incantation; cleansing each participant of the CBD production; and distributing marijuana in one or more containers. In one embodiment, the moon phase begins on a full moon. In one embodiment, a first zodiac sign is considered prior to performing the first biodynamic ritual. In one embodiment, the first biodynamic ritual comprises lighting bundles of sage. In another embodiment, participants of the biodynamic ritual are cleansed. In one embodiment, the moon phase is any one of: waning crescent, third quarter, waning gibbous, full moon, waxing gibbous, first quarter; and waxing crescent.

With this understanding, it is to be understood that the predictable orbit of the moon may influence the ebb-and-flow of bodies of water around the earth, including the oceanic tides. Considering that plants are comprised of mostly water, plants may be effected by the moon. Indeed, even the soil in which the plant resides may either retain water in a superior manner or dry out more rapidly, depending on the gravitational pull of the moon. Similarly, seeds may also respond by sprouting sooner and creating more robust plants when the moon is in a waxing state. Furthermore, it is believed that the effects of the moon's position in the zodiac may be partially explained by differences in electromagnetic currents transmitted by each respective planet.

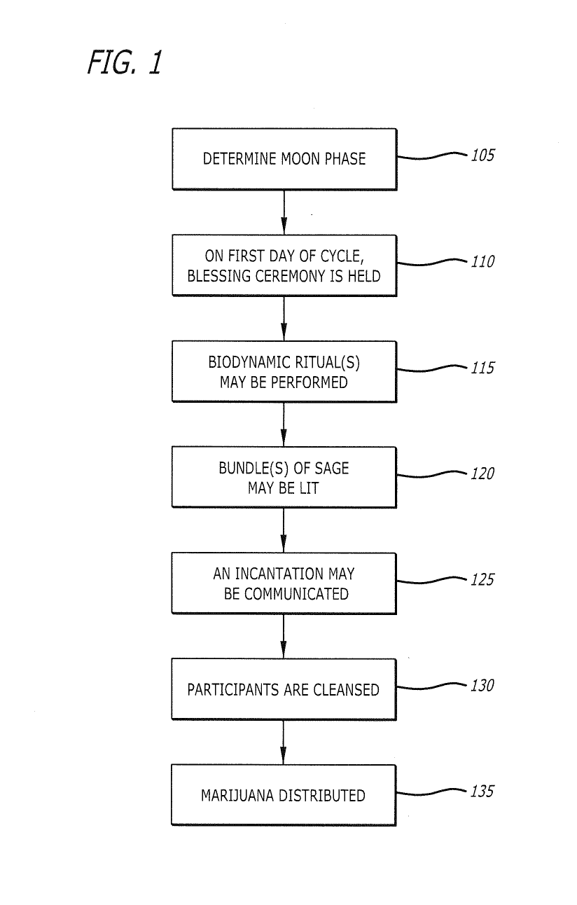

Referring now to FIG. 1, according to one embodiment, as shown in step 105, the moon's phase the moon is determined. It will be understood that it takes approximately 29.5 days for the phases to complete four stages of the lunar cycle. In one embodiment, a “New Moon” occurs when the moon is roughly in the same direction as the sun. In other words, the moon's illuminated half faces away from the Earth, and therefore the part that faces the Earth is not visible. Accordingly, the sun, Earth and moon align in almost a straight line, with the moon in between the sun and the Earth.

In step 110, on the first day of the cycle, a biodynamic ritual may be held under the stars so as to bless a work environment, such as a table. In step 115, a ritual may be performed including appreciation with respect to a Creator God and Mother Goddess. In step 120, depending on the product, a bundle of sage may be lit, and swept over any plurality of bottles and ingredients that include a high-proof alcohol to dissolve the bud. In step 125, a plurality of incantations may also be communicated. In step 130, the producers may utilize a bundle of sage so as to cleanse one another prior to production.

Finally, in step 135, in one embodiment, appropriate dosages of marijuana in various forms may be disposed into one or more preparation jars so that one or more CBD derived products may be produced. It will be appreciated that to maximize the amount of CBD during an extraction, cannabis varieties with high CBD content are preferred. The type of CBD extraction is crucial to getting good quality CBD extracts. In one embodiment, a solvent-based extraction method may be utilized, wherein a cannabis plant is submerged in a plurality of solvents. Thereafter, the resulting concentrate may be strained of plant matter and solvents. It should be appreciated that this method requires great caution and precision. In one embodiment, a CO2 extraction method may be implemented, wherein carbon dioxide is subjected to high pressure and very low temperatures so as to produce the concentrate. In one embodiment, olive oil may be infused with a CBD-rich plant so as to produce a CBD concentrate.

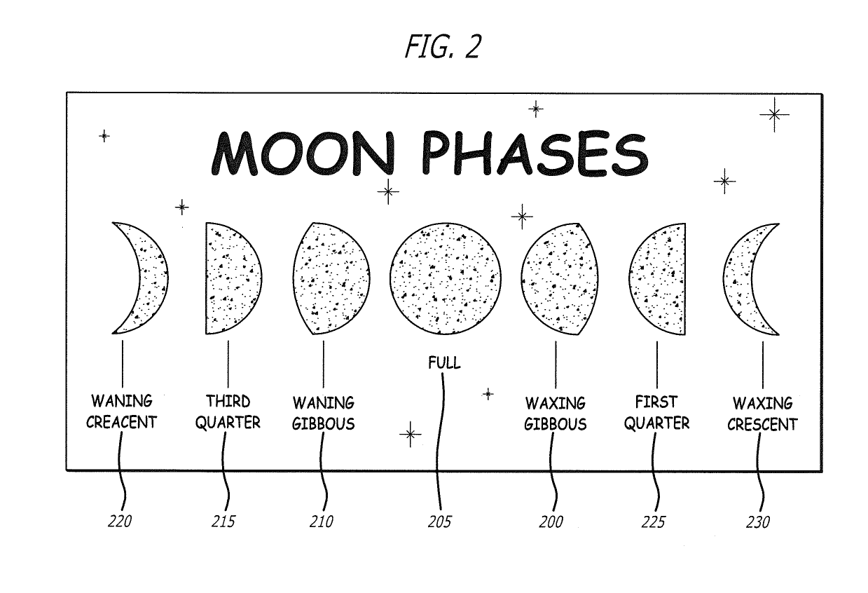

Referring now to FIG. 2, as the moon orbits about the Earth, the illuminated half may be more visible, and say the moon is in a “waxing” stage. At first, only a sliver of the moon is visible, which grows as days go by. This phase is known as the Crescent Moon. A week after the new moon, when the moon has completed about a quarter of its turn around the Earth, half of the illuminated part may be viewed. In other words, a quarter of the moon is visible, and thus it is referred to as the first quarter phase. During the next week, more of the illuminated part of the Moon may be seen, and it is now referred to as a waxing Gibbous (humped) 200 Two weeks after the new moon, the moon is halfway through its revolution. Now, it is to be understood that the illuminated half coincides with the one facing Earth, so that a full disc is substantially visible, which is referred to as a full moon 205. At this time, the moon rises when the sun sets, and sets when the sun rises. If the moon happens to align exactly with the Earth and sun, then a lunar eclipse is established.

Thereafter, until it becomes new again, the illuminated part of the moon decreases and it is referred to as waning. The first week after the full moon, the moon wanes as a waning Gibbous moon 210. Three weeks after the new moon, again half of the illuminated part of the moon may be viewed. Finally, during the fourth week, the moon is reduced to a thin sliver. The phase repeats with the new moon following the waning crescent moon 220.

With regard to the lunar cycle, the moon advances through four quarters, which includes the previous phases one through eight. The first quarter 225 begins with no visible moon moving into new moon, which, in some embodiments, may be marked by a small crescent. At this stage, moisture is at its peak, so this is generally regarded as a fertile time to take cuttings and to plant in an effort to produce high quality CBD derived products. More importantly, the growth of the roots and the leaves are in balance.

The second quarter begins approximately seven days after the new moon, ending with the full moon 205. From the new moon to the full moon 205 is a period of waxing, characterized by increased, sometimes rapid, growth and fertility, such as the during the waxing crescent 230. The third quarter 215 of the lunar cycle begins with the full moon 205, which, as the name implies, may be considered as a time of completion. In one embodiment, a biodynamic ritual may be performed to celebrate the fruits of the harvesters' labor by harvesting under the glow of the full moon. While the full moon 205 brings a peak in moisture levels similar to the new moon, in one embodiment, it is generally not recommended to plant on the full moon 205, as the subsequent waning in light and gravitational pull diminishes growth in the leaves. In one embodiment, during the fourth quarter of the moon's cycle, energy may be waning, and thus, the growth energy is pulled down into the roots. During this quarter of the moon's cycle, planting is not recommended, but general maintenance may be perfoiined. In one embodiment, during this period, plants may be watered, fertilized, and pest management may be implemented. In one embodiment, a biodynamic ritual may be performed during the third and fourth quarters to celebrate rest and reflection.

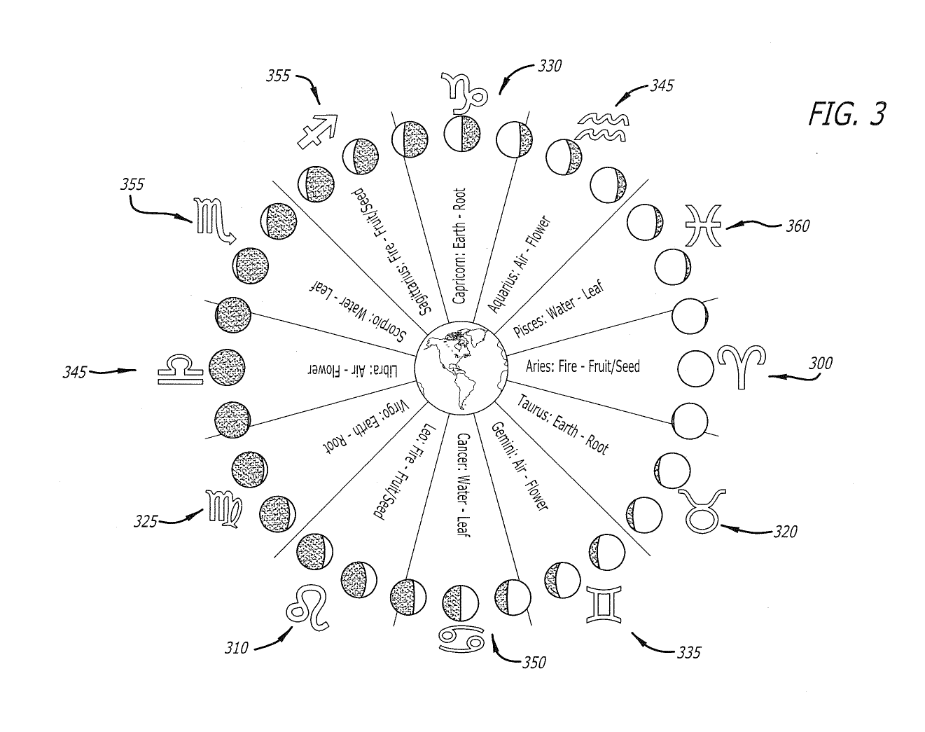

Referring now to FIG. 3, applying the lunar cycle also takes into account the planets and the twelve zodiac signs. In one embodiment, the moon spends approximately two-and-one-third days in each zodiac sign. Accordingly, the moon travels through all twelve zodiac signs substantially every twenty-eight days. It is envisioned that each zodiac sign features qualities of one of each of our four earthly elements: earth, air, fire and water. In one embodiment, each element may also be given masculine or feminine attributes. In one embodiment, dry, hot, barren attributes are considered masculine. In one embodiment, moist, cool, fertile attributes are considered feminine.

Referring still to FIG. 3, Aries 300 is a fire element, featuring one or more masculine attributes. When the moon is in Aries 300, it may be a good time to check plants for bugs, mold or yellowing leaves. However, this is not a recommended time for planting or starting seeds. The same holds true for Leo 310. On the other hand, while Sagittarius 315 is also not a recommended time for planting, it is fine to cultivate and harvest marijuana so as to produce CBD derived products. Moving now to Taurus 320, this zodiac sign features certain feminine qualities as an Earth element. With the lunar movement in this sign, plants are fertile. Accordingly, during orbit through a Taurus 320 sign, plants may respond especially well to planting. On the other hand, while Virgo 325 features moist and feminine Earth qualities, it is a relatively barren time better left to removing pests such as with respect to Aries 300, rather than planting seeds. Capricorn 330, the final earth sign, is more productive than Virgo 325, but it is not generally considered to be as productive as Taurus 320.

Still with respect to FIG. 3, Gemini 335 is dry and barren due to its airy and masculine qualities. During the Gemini 335 phase, transplanting or planting is not recommended, but general maintenance is fine. Similarly, although primarily dry and barren, Libra 340 features enough moisture to encourage fertility and strong root growth, and is thus a good period of time for transplanting. Aquarius 345, while not fertile, may be a good time to harvest the crops. The water signs: Cancer 350, Scorpio 355 and Pisces 360 are all generally considered to be feminine signs. Consequently, these three zodiac signs are understood to be “happy times” for successful growing of marijuana. In some embodiments, when the moon is in Cancer, it may be ideal to take cuttings, plant seeds and transplant as necessary. During Scorpio 355, a productive and abundant time for planting may be observed.

In one embodiment, the moon cycles may be utilized so as to develop any of a plurality of CBD derived products, including but not limited to topical salves, tinctures, and oils made from hemp extract, so as to provide relief from pain, PTSD, debilitating diseases, and more. In one embodiment, the cannabis salve may be used to heal back pain, arthritis and erase scar tissue. The salve may be made from a combination of cannabis trim, coconut oil, vitamin E, lavender oil, calendula oil, and beeswax in varying quantities. In one embodiment, high-CBD cannabis tincture may be turned every morning and night, according to one biodynamic ritual. In one embodiment, the CBD tincture is only bottled during a full moon, after the moon cycle completes. In one embodiment, CBD oil is comprised of 97% pure CBD crystalline. In one embodiment, the CBD products may be organic, lab-tested and pesticide-free, or any combination thereof.

In one embodiment, the CBD derived products are only manufactured from the new moon to the full moon. On the first day of the cycle, a biodynamic ritual may be held under the stars so as to bless a work environment, such as a table. The ritual may include appreciation with respect to a Creator God and Mother Goddess. Depending on the product, a bundle of sage may be lit, and swept over any plurality of bottles and ingredients that include a high-proof alcohol to dissolve the bud. A plurality of incantations may also be communicated. In one embodiment, the producers may utilize a bundle of sage so as to cleanse one another prior to production. In one embodiment, appropriate dosages of marijuana may be scooped into preparation jars.

In one embodiment, a Palo Santo stick may be utilized in one or more biodynamic ritual. It is to be appreciated that the smell of the wood when burned may induce happiness in the preparer. In one embodiment, to brew a fresh batch of salve, in conjunction with a biodynamic ritual, first gloves are worn on the hands, and preparations begin. During a first step of preparation, candles may be lit, sage may be burned, and soft music may be played. In one embodiment, music or chanting indicating that “The earth is our mother, we must take care of her” may be repeated. Prior to distributing the ingredients into a plurality of pots, a prayer huddle may be performed.

During the prayer huddle, if multiple preparers are present, hands may be held and everyone's eyes may be closed. In one embodiment, one of the preparers may indicate appreciation by chanting “Thank you for calling us to do this divine work,” preferably softly and slowly, with conviction. In one embodiment of the biodynamic ritual, one may chant “Let our medicine travel safely and do the work it needs to, and then let the people using our medicine do the work they need to do. Keep our hearts pure and their hearts pure. Keep our minds pure and our hands steady.” Preferably, one or more vows may be taken, including for example, servitude, activism, obedience, ecology, poverty. More specifically, during the two weeks from new moon until full moon, veganism and celibacy may be undertaken.

In one embodiment, pure plant CBD oil may be composed using CO2 extraction methods, so as to comprise substantially 360 mg of CBD per gram of oil. In one embodiment, topical salve may be implemented using the aforementioned techniques so as to assist carrying the CBD compounds into one's system through the skin. In some embodiments, it is envisioned that there will be no difference between the potency of one or more CBD-infused oils and CBD tinctures. It should be appreciated that both CBD-infused oils and CBD tinctures are mediums of getting the medicine into the system via drops on the tongue. In one embodiment, the CBD infused oil is comprised from liquid coconut oil and may have a mild taste. The tincture, however, is comprised of food-grade high-grain alcohol and high-CBD organic plant.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A method for producing CBD-derived products, comprising:
determining an instance of a new moon;
planting cannabis cuttings during a beginning of a first quarter of the lunar cycle;
performing at least one biodynamic ritual during a third quarter of the lunar cycle;
maintaining cannabis plants during a fourth quarter of the lunar cycle;
celebrating rest and reflection through one or more biodynamic rituals during the third quarter and the fourth quarter; and
harvesting mature cannabis plants under the glow of the full moon during the third quarter.

2. The method of claim 1, wherein maintaining cannabis plants includes implementing any of watering, fertilizing, and managing pests.

3. The method of claim 1, further comprising extracting a CBD concentrate from harvested mature cannabis plants.

4. The method of claim 3, wherein extracting includes submerging cannabis in one or more suitable solvents and straining of plant matter and solvents to produce the CBD concentrate.

5. The method of claim 3, wherein extracting includes subjecting the cannabis to carbon dioxide under high pressure and very low temperature and straining of plant matter to produce the CBD concentrate.

6. The method of claim 3, wherein extracting includes infusing the cannabis with olive oil and straining of plant matter to produce the CBD concentrate.

7. The method of claim 3, further comprising incorporating a suitable quantity of the CBD concentration into any of a plurality of desired CBD-based products.

8. The method of claim 1, wherein determining includes correlating the lunar cycle with the twelve zodiac signs so as to guide in the cultivation and harvest of the mature cannabis plants.
